(19) United States
(12) Patent Application Publication
(10) Pub. No.: US 2017/0209103 A1
(43) Pub. Date: Jul. 27, 2017

(54) Simplified Instances of Virtual Physiological Systems for Internet of Things Processing

(71) Applicant: LifeQ Global Limited, Dublin (IE)

(72) Inventors: Laurence Richard Olivier, Alpharetta, GA (US); Franco Bauer du Preez, Cobham (GB); Riaan Conradie, Alpharetta, GA (US); Gerrida Mathilda Uys, Oranjezicht (ZA)

(21) Appl. No.: 15/415,443

(22) Filed: Jan. 25, 2017

Related U.S. Application Data
(60) Provisional application No. 62/286,577, filed on Jan. 25, 2016.

(51) Int. Cl. A61B 5/00; G06F 19/00; G06Q 40/08; A61B 5/0205; A61B 5/145

(52) U.S. Cl. CPC A61B 5/7278; A61B 5/02055; A61B 5/14542; G06Q 40/08; G06F 19/328; A61B 5/024

(57) ABSTRACT

The claimed invention presents methods for predicting the outcomes of physiological systems in real time using limited data input and computational resources. The claimed invention is comprised of simplified, or abstracted, physiological models, derived from detailed cloud-based computational systems biology models. Abstracted models are capable of utilizing limited data streams, typically obtained from non-invasive data acquisition devices, to accurately estimate, predict and display the outcomes of physiological systems in real time on the device, compared to detailed cloud-based estimations that are computationally demanding and can be used to continuously update abstracted models over time.

### CROSS REFERENCE TO RELATED PATENT APPLICATIONS

This invention claims priority from U.S. Provisional Patent Application No. 62/286,577, filed Jan. 25, 2016, which is hereby incorporated by reference.

### FIELD OF INVENTION

The present invention relates to the field of non-invasive physiological monitoring and computation of biological data. More specifically, methods are presented for predicting the outcomes of physiological systems in real time using limited data input and computational resources.

### BACKGROUND OF INVENTION

Techniques for generating personalized biological information are outpacing Moore's law on electronics. For example, DNA sequencing technologies are currently developing at supra-exponential rates to deliver full genome information at the consumer level in single years from the present. The rapid rate of generating such vast amounts of biological information, however, far exceeds the rate of generated information being processed and interpreted by research and clinical communities, especially in consumer and patient-relevant contexts.

The emergence of mainstream wearable technology has led to the development of a vast array of sensors capable of continuously monitoring physiological signals in non-invasive ways. Using these sensors and sensor-derived data in combination with computing devices and internet communications opens up possibilities to bring the human body to the Internet of Things (IoT). The majority of current wearable and mobile devices can generate personal health data streams and metrics, communicate said data and metrics to internet databases which can then create health ecosystems that allow the a subject to manage and improve his or her personal wellbeing. The speed and accuracy of computing personal health metrics from data streams obtained from wearable devices are continuously being improved by the development of more sophisticated sensors and algorithms. Personal health data and metrics, together with a user-enabled holistic health management approach, may contribute to models of prevention and possibly diagnostics. However, challenges remain to make accurate and biologically relevant inferences and predictions from non-invasive physiological signals and subsequent information flows that are valuable to the consumer, clinician and researcher alike.

Significant progress has been made in recent years towards the quantitative modelling of the human body. The scientific field of computational systems biology (CSB) aims to capture and predict the behaviour of biological systems and expand on understanding these systems using mathematical models that describe the behaviour of the different systems that work together to generate the human body's emergent behaviours. For example, known human models include, but are not limited to, respiratory, brain, cardiac and liver models. Knowledge of these biological systems can be captured in computable format using quantitative modelling. Moreover, models can be used in conjunction with each other or in conjunction with other types of mathematical models (i.e., probabilistic models).

The present invention aims to address the need for biologically and clinically relevant inferences and predictions computed from limited data streams, typically obtained by non-invasive devices such as wearables.

### SUMMARY OF THE INVENTION

The claimed invention aims to provide methods for accurately predicting and inferring difficult to measure physiological parameters utilizing limited data streams, such as those typically acquired by non-invasive devices (e.g., subject-wearable data acquisition devices). In an aspect, abstracted versions of detailed and demanding computational systems biology (CSB) models of physiological systems are communicated to data acquisition devices in immediate vicinities of data acquisition sensors, to enable real time estimations and display of complex physiological parameters of the subject on the device. In an aspect, these abstracted models are capable of utilizing limited data streams, to accurately estimate, predict, and display the outcomes of physiological systems in real time on the device, compared to detailed cloud-based estimations that are computationally demanding and continuously updated over time. In an aspect, the non-invasive data acquisition devices can provide the limited data streams utilized by the abstract models to produce the outcomes.

In an aspect, the claimed invention utilizes a two-part CSB modeling approach. In the first part, multiple detailed and computationally demanding CSB models, typically hosted via cloud computing resources, are used in combination with each other to create virtual physiological systems. In an aspect, probabilistic models can also be used in combination with the CSB models to generate the virtual physiological systems. In exemplary aspects, probabilistic models can form an interface between the CSB models and measured data in order to optimize the mapping of measured parameters to those parameters inferred from physiological systems. Biological, demographic, and database metrics of a subject are used as input for virtual physiological systems to enable personalized probabilistic modeling of physiological parameters that is updated and modeled over time. This type of modeling enables quantitative descriptions of a user's physiology and behavior. From the virtual physiological systems, abstracted versions can be created, which are more simplified and hence less computationally complex for peripheral processing in wearable devices with limited processing capabilities and energy storage.

In an aspect, abstracted versions of these personalized virtual physiological systems are regularly communicated to processing hardware in more immediate vicinities of data acquisition devices associated with the subject. The abstract models, derived from the detailed cloud-based models, generate approximately the same output as detailed models, but utilize limited data streams as input, and modeling said output in real time. By utilizing abstracted physiological models on data acquisition devices, immediate and easily accessible measurements (e.g., example heart rate, oxygen saturation and breathing rate) are employed to estimate physiological parameters for the subject that are less accessible and difficult to measure. For example, the metabolic rate, respiratory quotient, heart stroke volume, hematocrit levels, and/or arterial and venous oxygen difference of the subject can be generated by the abstracted physiological models on the local data acquisition devices through the accessible measurements. Abstracted models require less computing power than detailed models, and can be regularly communicated via wireless technology to processing hardware in more immediate vicinities of data acquisition device (e.g., subject's wearable device). Estimations of less accessible physiological parameters therefore occur on the data acquisition device itself and can be displayed in real time.

The claimed invention presents methods for real time and accurate estimation and prediction of complex physiological parameters from limited data streams, typically obtained by non-invasive devices exemplified by, but not limited to, wearable devices. These and other aspects of the invention are realized from a reading and understanding of the detailed description and drawings.

### BRIEF DESCRIPTION OF DRAWINGS

FIG. 1 is a schematic representation of a virtual physiology ecosystem illustrating various embodiments of the claimed invention.

FIG. 2 is a schematic representation of a wiring diagram of a computational system biological model according to an aspect of the present invention.

### DETAILED DESCRIPTION OF INVENTION AND DRAWINGS

Herewith, the detailed description and drawings explain varying aspects of the present invention. The description and drawings serve to aid one skilled in the art to fully understand the present invention and are not by any means intended to limit the scope of the invention. Before the present method and system are disclosed and described, it is to be understood that the method and system are not limited to special methods, special components, or to particular implementations. It is to be understood that the terminology used here is for the purpose of describing particular aspects only and it is not intended to be restrictive. As used in the specification and the appended claims, the word “comprise” and variances of the word such as “comprising” and “comprises”, means including, but not limited to, and are not intended to exclude, for example, other components or steps. “Exemplary” means “an example of” and it is not intended to convey an indication of a preferred or ideal embodiment. “Such as” is not used in a restrictive sense, but for explanatory purposes. The singular forms “a”, “an” and “the” also include plural elements unless the content clearly dictates otherwise.

In an aspect, the systems discussed above and below utilize computer processing in order to generate the various models and data. Further, one skilled in the art will appreciate that the systems and methods disclosed herein can utilize various computing devices, including, a general-purpose computing devices, cloud-based servers, and various other computing means known in the art. The various computing devices discussed below perform their duties and responsibilities through the use of processors or processing units, human interfaces, system memory, storage means, operating systems, software, data, network adapters, wireless transceivers, interfaces, and the like.

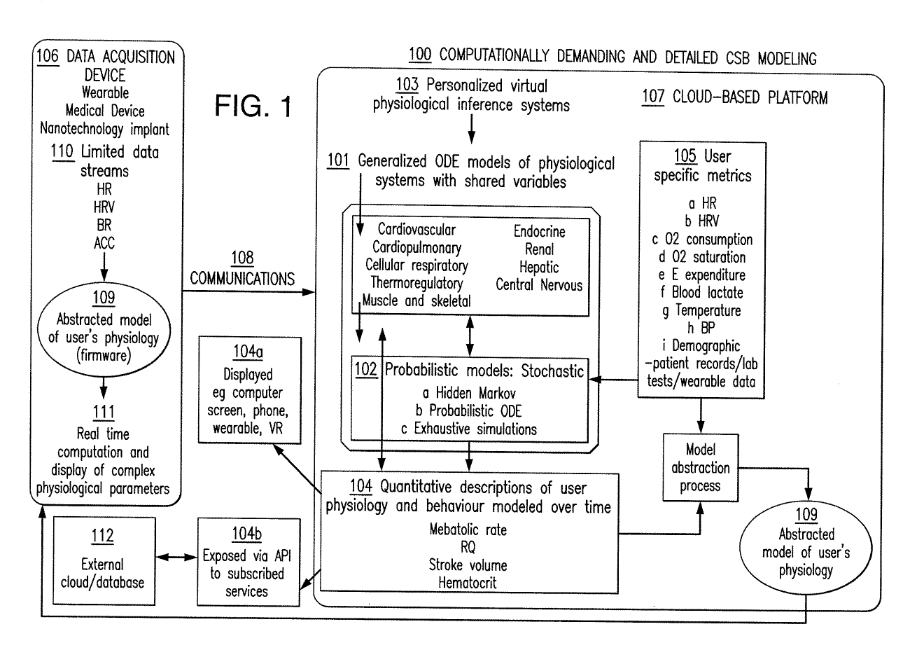

In an aspect, the invention is aimed at providing more immediately accessible physiological parameters through the use of a two-part computational system 100 that utilizes computationally demanding and detailed computational systems biology (CSB) modelling in an abstract form to provide information to the subject. In an aspect, the claimed invention utilizes a two-part CSB modelling approach. In the first part, detailed and computationally demanding CSB models 101, typically hosted via cloud computing resources 106, are used in combination with one another (e.g., cardiovascular with cardiopulmonary, as listed in FIG. 1) to build virtual cloud-based physiological systems 103. In an aspect, the CSB models 101 are comprised of generalized ODE models of physiological systems with shared variables. In an aspect, the CSB models 101 can include, but are not limited to, models generated to represent cardiovascular, cardiopulmonary, cellular respiratory, thermoregulatory, muscle and skeletal, endocrine, renal, hepatic, and central nervous systems. Other examples of CSB models 101 are found in co-pending PCT Application No. PCT/US2015/043919, titled Biologically Inspired Motion Compensation and Real-Time physiological Load Estimation Using a Dynamic Heart Rate Prediction Model, filed Aug. 6, 2015, and incorporated herein by reference in its entirety. In an aspect, these virtual physiological systems 103 are inference-based.

User specific metrics 105 serve as input for said cloud-based physiological systems 103, with the utilization of probabilistic models 102, enables the physiological systems 103 to generate personalized estimations and inferences of physiological parameter sets and quantitative descriptions 104 of a specific user's physiology and behavior, which are updated and modeled over time. In an aspect, the probabilistic models 102 can be stochastic models 102. In such instances, the probabilistic models can include, but are not limited to, hidden Markov models 102a, probabilistic ODE models 102b, and exhaustive simulation models 102c. Further, the user specific metrics 105 can include, but are not limited to, hear rate 105a, HRV 105b, oxygen consumption 105c, oxygen saturation 105d, E expenditure 105e, blood lactate 105f, temperature 105g, blood pressure 105h, and demographic information 105i. For demographic data 105i, that the ranges for these values could be calibrated using other digital health data sources including patient records, lab tests and wearables.

For the above combinations (CSB models 101, probabilistic models 102, and user metrics 105), personalized virtual physiological systems 103 can be generated. These systems 103 can then generate physiological parameter sets and quantitative descriptions 104. Examples of the physiological parameter sets and quantitative descriptions 104 include, but are not limited to, a subject's metabolic rate, respiratory quotient, heart stroke volume, hematocrit levels, and arterial and venous oxygen difference.

In the second part, abstracted versions 109 of said virtual physiological models 103 are regularly communicated via wireless technology 108 to processing hardware in more immediate vicinities of the subject and data acquisition sensors (e.g., the hardware found on the data acquisition device 106 or a mobile device associated with the subject that is in communication with said sensors). Immediate and easily measured physiological parameters 110, typically acquired by non-invasive data acquisition devices 106, subsequently serve as direct data input for abstracted models 109 that are employed to estimate less accessible and more difficult to measure physiological parameters 111 on the device 106 in real time. The claimed invention presents methods by which more immediately accessible physiological parameters 110, exemplified by, but not limited to, heart rate, oxygen saturation and breathing rate can be employed to estimate physiological parameters 111 that are less accessible, for example, but not limited to, a subject's metabolic rate, respiratory quotient, heart stroke volume, hematocrit levels and arterial and venous oxygen difference.

In an aspect, as illustrated in FIG. 1, the two part computational system utilizes a combination of a cloud based platform 107, configured to communicate over various communication means 108, with a remote data acquisition device 106 (or, in some instances, a remote computing device in communication with the data acquisition device 106) closer proximity to the subject for which the physiological parameters are generated. The cloud based platform 107 and the data acquisition device 106 work in conjunction with one another to provide the physiological parameters to the subject via the data acquisition device 106, discussed in further detail below.

### Detailed Cloud-based Models

Unlike a controlled experiment, a subject's physiology is subject to behavioral choices that have an impact on the subject's physiological parameters. For example, the choice of a subject to go running can change that person's heart rate by a factor of three in some instances, depending on the subject's health and intensity at which the subject runs. Given the impact of a subject's behavior on physiological parameters, there is a need for a systematic description of this uncertainty in the form of a probabilistic model of user behavior, as well as a framework for calculating the most likely trajectory of said subject's physiology. This is achieved by considering user physiology and behavior simultaneously to explain continuous metric feeds into cloud modeled physiological systems.

In an aspect, as illustrated in FIG. 1, virtual physiological systems 103 run remotely on a cloud-based platform 107. Virtual physiological systems 103 can be created on the cloud based platform 107 through the use of interconnecting modules describing different physiological: Generalized CSB models 101, together with probabilistic models 102, for creating a personalized virtual physiological system 103 to infer a user's most likely physiological history and/or behaviour to generate physiological parameter sets and quantitative descriptions 104 based on said user's continuously updated metrics 105. The data 104 can be displayed in some instances to a subject through a computer or wearable device 104a. In addition, the data 104 can be supplied to external databases 112 via APIs 104b.

The virtual physiological system 103 can acquire additional information (e.g., demographics 105i from external cloud services and databases 112) via various APIs 104b. In an aspect, external factors that drive physiology, such as exercise intensity, is inferred by the probabilistic inference layer 102 in combination with the models 101. In another aspect, a large number of alternative hypothesis explaining the observed physiology as seen in the wearable data is tried out and the most likely exercise level, or muscle load, is continuously inferred as external parameters that affects the virtual physiology and brings it in line with the real physiology. In an aspect, the data acquisition device 106 can supply the user specific metrics 105. In other aspects, the other devices can supply information (e.g., demographics 105i). Probabilistic models 102 are exemplified by, but not limited to, stochastic models of user behaviour, Hidden Markov Models (HMM) and exhaustive simulations.

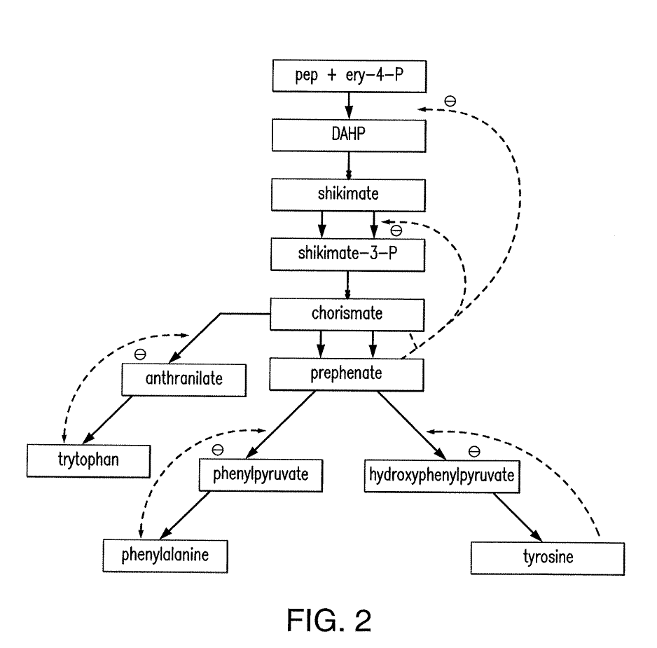

In particular embodiments, ordinary differential equations (ODEs) are used to describe CSB wiring diagrams (FIG. 2 illustrates an example) describing physiological systems 103 that, according to current experimental knowledge, best describes the biological system (e.g., cardiac system, pulmonary system, etc.) of the subject. In an aspect, the probabilistic inference system 103 infers the most likely state of external stochastic factors such as degree of exercise/posture/fever and applies it to the system 103 to match the virtual parameter outputs to the real parameter outputs, while generating predictions for external factors. At the same time the model simulation can also be used to get predictions for internal parameters that are not available from wearable sensors such as blood pressure, which is explicitly part of the system being simulated. ODEs describes how processes within a system affect the rate of change of a variable:

$$\frac{dX}{dt} = {\sum }_{i = 1}^{p} {v}_{{production}_{i}} - {\sum }_{i = 1}^{c} {v}_{{consumption}_{i}}$$

where the rates of the processes v are summed for the total number (p) of processes producing X, subtracted by the total number (c) of processes consuming X The processes affecting the variables of a biological system can be biochemical or biophysical in nature. For example, biochemical reactions include the oxidation of macronutrients to produce water and carbon dioxide and can be translated to energy expenditure, while biophysical reactions include phenomena such as the variation in pressure in the aorta due to its elasticity, peripheral vascular impedance and the injection of a volume of blood (heart stroke volume) every time the heart contracts.

Specific sets of ODEs (e.g., those pertaining to cardiovascular and pulmonary physiology, heat exchange, and endocrine functions) are used to describe CSB wiring diagrams of physiological systems 101, and model parameters are fit on experimental observations. In an aspect, the model parameters can include measurable parameters (e.g., but not limited to, heart rate) and internal parameters (e.g., but not limited to, blood pressure in the aorta). In an aspect, experimental parameters can be collected from various sources, including published experiments, information gathered in trials, and information supplied by partners. If the set of ODEs fails to describe the experimental observations (first qualitatively and then quantitatively), another set of ODEs are adapted, followed by further parameter fitting until the set of ODEs can accurately describe the experimental observations as per normal physiology as well as pathophysiology.

In preferred embodiments, sets of generalized ODE models with shared variables are combined to construct a cloud-based virtual physiological system 101. Examples of ODE models with shared variables that are combined to construct virtual physiological systems include, but are not limited to, models of cardiovascular systems, cardiopulmonary systems, cellular respiratory systems, thermoregulatory systems, endocrine systems, renal systems, hepatic systems, skeletal and muscle systems, and central nervous systems. Additional examples of these systems can be found at www.physiome.org.

User specific metrics 105 exemplified by, but not limited to, database metrics, biological metrics and demographic data serve as input to enable probalistic modelling 102 of user physiology and behaviour by utilizing stochastic models such as Hidden Markov models (HMM) and/or exhaustive simulations in parallel with predictive ODE models. As discussed above, the information can be provided through various devices. Probabilistic modelling 102 from virtual physiological systems 103 using user specific metrics 105 is a continuous process requiring heavy computing power, and may occur over time, and may be frequently or infrequently updated with either newly acquired biological or database user specific metrics 105. Personalized parameter sets and quantitative descriptions 104 of a specific user's physiology and behaviour are generated by probabilistic modelling 102 and generalized CSB models 101 together with biological, database and demographic input 105.

### Data Acquisition for Model Input

In particular embodiments, data required for metric computation that serves as input for generalized CSB models 101 and/or probabilistic models 102 may be acquired in the following ways: A user's physiological data streams 110 are acquired utilizing data acquisition devices 106 capable of communicating said acquired physiological data streams 110 to a computing device/cloud-based platform 107 capable of communicating over various communication means 108 including, but not limited to, wireless networks, the interne, and various other methods and combinations thereof Examples of data acquisition devices 106 include, but are not limited to, wearable devices, medical devices, implants and nanotechnology. In an aspect, the data acquisition device can include, but is not limited to, the wearable data acquisition device disclosed in U.S. patent application Ser. No. 14/128,675, incorporated in its entirety by reference. Physiological data streams 110 may be comprised of one or a combination of the following: cardiac signals, pulmonary signals, motion signals, electrodermal signals, thermal signals, blood signals and brain signals. The data acquisition device 106 can utilize various sensors known in the art to collect and generate such signals. Environmental measurements obtained from data acquisition devices, for example outside temperature, may also serve as data streams 110. In an aspect, physiological data streams 110 are communicated from the data acquisition device 106 to a computing device. In exemplary aspects, the computing device can be combined with the data acquisition device 106. In an aspect, the computer device is configured to process the data streams 110. In an aspect, the data streams 110 can be subject to digital signal and algorithm processing. The data streams 110 are processed into biological metrics 105 for transmission through the communications means 108 to a cloud-based platform 107. Alternatively, digital signal and algorithm processing of physiological data streams into biological metrics 105 occur on a stand-alone computing device, followed by communications of said metrics to a cloud-based platform 107. In other embodiments, physiological data streams 110 are communicated from the data acquisition device 106 and/or computing device directly to a cloud-based platform 107, followed by digital signal and algorithm processing of said data streams into biological metrics on the cloud-based platform.

Examples of biological metrics 105 include, but are not limited to, heart rate 105a, heart rate variability 105b, oxygen consumption 105c, oxygen saturation 105d, energy expenditure 105e, blood lactate values 105f, body temperature 105g and blood pressure 105e. Biological metrics 105 serve as primary input for probabilistic modelling 102, and may be frequently and/or continuously updated as new physiological data streams 110 are acquired. The continuous updating leads to a frequent and/or continuous feed of biological metric input 105 to the cloud-based models 101, 102, enabling frequently informed or live virtual estimations and/or inferences of physiological parameters 103. Demographic data 105i may also serve as input for detailed CSB modelling 101 and/or probabilistic modelling 102. Demographic data includes, but is not limited to, a user's age, sex and ethnicity.

In other embodiments, subject data may be acquired from existing external databases 112. Existing databases may include one or a combination of the following: medical, genetic, proteomic, environmental, genealogical, epidemiological, population, psychiatric, behavioural and family history databases. Information acquired from said databases 112 are processed into metrics 105 on a computing device connected to a cloud-based platform 107, followed by communication 108, 104b of said metrics to the cloud-based platform 107. Alternatively, information from databases are communicated directly from database servers to cloud-based platforms 107 followed by cloud computing of information into metrics 105. Metrics computed from data acquired from said databases 112 (from here referred to as database metrics) serve as secondary input into probabilistic modelling 102, and may be updated to enable frequently informed or live virtual estimations and/or inferences of physiological parameters 103.

By utilizing a user's demographic, biological and database metrics 105 as input together with probabilistic modelling 102, generalized CSB models 103 of virtual physiological systems 101 are capable of generating personalized parameter sets and quantitative descriptions 104 of a specific user's physiology. Many of these parameters 104 can be estimated by varying underlying parameters in the models 101 to see which virtual physiology system 103 matches the collected data best—this cannot be done in isolation because the body is a system where all parts interact to produce a behaviour—hence the need for a CSB approach where simulations are performed with all the relevant parts included. For example, internal model parameters such as aorta elasticity can also be adjusted in the model to similarly infer the most likely parameter value via the probabilistic inference layer for such an internal parameter. Other examples include, but are not limited to, inference of autonomic tone from heart rate variability and heart rate recovery data, aorta elasticity inference from PPG amplitude and waveform, heart stroke volume inference from metabolic rate (that could be inferred from eg heat flux sensors and body surface area (e.g., estimated from height and weight), and thermal conductivity from long term heart rate recovery pattern after exercise.

### Abstracted Models of Detailed Cloud-based Models

In particular embodiments, a user's physiology is modelled over time on a cloud-based platform 106 utilizing newly acquired and/or updated demographic, biological and database metrics 105. Personalized physiological parameter sets and quantitative descriptions 104 of a specific user's physiology are generated by the combination of CSB models 101 and probabilistic modelling 102, and represents a virtual physiological system 103 of said user on a cloud-based platform 107. This system 103 is then transformed into an abstract model 109. The abstract models 109 can then be run locally in relation to the subject. For example, the abstract model 109 can be stored on the data acquisition device 106. The abstract models 109 can then provide physiological parameters 111 to the subject through the data acquisition device directly, without having to call upon the cloud based platform 106.

A point is reached where abstracted models 109 can be derived from user specific detailed physiological models 103. In an aspect, a detailed physiological model 103 parameterized by wearable and demographic data can be simplified or abstracted 109 such that it maps wearable inputs to outputs of interest with a much reduced computational load and that it will remain aligned with the user's physiology for a limited time. User specific detailed physiological models 103 can be simplified, or abstracted, by example, but not limited to, linear models, polynomial, or simple ODE models 109 with a limited number of state variables and computational complexity, and stochastic inference models such as HMMs, that will yield approximately the same output as the detailed models 103, but using limited data streams as input. Examples of limited data streams 110 include, but are not limited to, one or a combination of the following: heart rate, breathing rate, temperature and accelerometer data streams 110. In an aspect, the data acquisition device 106 can provide the data streams 110. Abstracted models 109 may be adjusted and/or updated as adjustments and/or updates are made to the detailed model. For example, new profile data can be provided, utilizing new data steams (e.g., weight from a connected scale), aging process that changes the stiffness of the aorta, and the like can occur. Newly constructed, adjusted or updated abstracted models 109 of a specific user are communicated via wireless communications 108 to computing device/s, exemplified by, but not limited to, said subject's wearable device 106, in close proximity to data acquisition sensors. Limited, but immediately accessible, data streams 110 serve as input for abstracted models 109 that enables real time computation and read-outs of complex and difficult to measure physiological parameters 111 on a computing and/or data acquisition device 106 in close proximity to the data acquisition sensors. Examples of complex and difficult to measure physiological parameters include, but are not limited to, a user's metabolic rate, respiratory quotient, heart stroke volume and hematocrit levels. This enables a temporary linearization of physiology which can be updated intermittently as physiology changes.

### Use Case—Abstracted Model to Estimate Respiratory Quotient (RQ)

The respiratory quotient (RQ) value indicates the ratio of carbon dioxide molecules produced per oxygen molecules consumed by aggregate metabolic processes in the body, and is calculated with the formula: RQ=carbon dioxide eliminated/oxygen consumed. The RQ value varies according to the chemical constitution of the nutrients on which a person relies for energy production. In the case of fats, only 0.7 molecules of carbon dioxide are produced per oxygen molecule consumed by metabolism, while this is closer to a 1:1 ratio when carbohydrates are consumed. RQ values are typically measured by complicated sports performance laboratory equipment such as an indirect calorimeter.

RQ is therefore a complicated physiological parameter 111 that can be quantitatively measured. This enables accurate validation of RQ values inferred from detailed and abstracted models 109 against laboratory-grade measurements. In some embodiments, an integrated cloud-based physiological model 101 is set up, by combining ODE models with shared variables, exemplified by models of cardiopulmonary physiology, blood gases, tissue metabolism and homeostatic control of heart and breathing rate. User specific biological metrics 105, exemplified by heart rate, oxygen consumption, oxygen saturation, energy expenditure and blood lactate values serves as input for the integrated cloud-based physiological model.

By simulating the detailed physiological model 109 over a broad range of exercise and dietary perturbations, by adjusting both the RQ of the energy sources supplied and the level of tissue metabolism, it is possible to obtain steady state heart rate and ventilation rate predictions 111 from the model. This process of exhaustive simulation of the model over a range of internal states that are targeted to be inferred, creates a mapping (i.e., the abstract models 109) from values of the internal states that cannot be directly measured to external signals that can be monitored. This mapping can be inverted mathematically and summarized as a reduced or ‘linearized’ model that generate an estimate for metabolic rate and RQ 111 given heart rate and ventilation rate data streams 110, which may be validated against actual laboratory measurements to determine accuracy. In short, non-invasive measurements, exemplified by real time heart rate and ventilation rate 110, obtained from sensors in the wearable device 106, serve as direct input for the abstracted model 109, and enables real time calculations and display of a user's RQ value 111 on the device 106.

Having thus described exemplary embodiments of a method to determine sleep stages and other related data, it should be noted by those skilled in the art that the within disclosures are exemplary only and that various other alternatives, adaptations, and modifications may be made within the scope of this disclosure. Accordingly, the invention is not limited to the specific embodiments as illustrated herein, but is only limited by the following claims.

What is claimed:

1. A method in which one or a combination of two or more accessible and/or easy to measure physiological parameters, exemplified by, but not limited to, heart rate, breathing rate, oxygen saturation, blood pressure and body temperature, are employed to estimate physiological parameters that are less accessible and/or difficult to measure, for example, but not limited to, metabolic rate and respiratory quotient, said method comprising:
a. One or a combination of two or more computational models describing a physiological system and/or sets of physiological systems
b. A process of generating a simplified, or abstracted, model from the predictions of the model in a. to map accessible physiological parameters to invasive, non-accessible physiological parameters
c. Using the model abstraction in b. on a more computationally constrained computing platform, including mobile or wearable devices, to gain mobility and real time feedback with regards to the estimated physiological parameter(s)

2. The method of claim 1, where the process of generating a simplified model comprises probabilistic modeling, including, but limited to, stochastic models, Hidden Markov models, probabilistic ODEs and exhaustive simulations, said modeling used to infer complex personalized physiological parameters, variables and states of a person's physiology from a given set of metric inputs, said metric inputs obtained from one or a combination of two or more physiological, clinical, genomic, proteomic, population, environmental and demographic metrics.

3. The method of claim 1, wherein abstracted models of physiological systems, or updated versions of said abstracted models, are communicated via wireless technologies from processing platforms of origin, exemplified by, but not limited to, cloud-based platforms, to processing hardware within either more immediate vicinities of physiological measuring instruments or processing hardware contained within measuring instruments, or both, thereby enabling continuous and/or real time estimations and display of complex physiological parameters on said instrument and/or connected device

4. The method of claim 1, where physiological inferences, obtained from quantitative descriptions of a person or population's physiology from a given set of metric inputs, or abstracted modeling from a limited set of inputs, are communicated via wireless technologies from processing platforms to third party databases, exemplified by, but not limited to, clinical, insurance, retail and biological databases.

5. A virtual physiology ecosystem utilizing a two-part computational system biological approach for generating models for a subject comprising:
a. a local computing device; and
b. cloud computing resources, wherein the cloud computing resources are configured to
i. generate at least one computational system biology (CSB) model of the subject;
ii. apply probabilistic models to the at CSB models to a generate virtual cloud-based physiological system of the subject;
iii. generate an abstract of the virtual cloud-based physiological system; and
iv. transfer the abstract model to the local computing device, wherein the local computing device is configure to take limited data streams from the subject and supply the data streams to the abstract model to generate real time physiological parameters of the subject.

6. The virtual physiology ecosystem of claim 5, wherein the virtual cloud-based physiological system is configured to make interferences and estimates of physiological parameter sets and quantitative descriptions of the subject.

7. The virtual physiology ecosystem of claim 6, wherein the probabilistic models utilize user specific metrics to generate the virtual cloud-based physiological system of the subject.

8. The virtual physiology ecosystem of claim 7, wherein the user specific metrics comprise demographic information.

9. The virtual physiology ecosystem of claim 5, wherein the cloud computing resources is configured to generate a plurality of CSB models and combine a portion of the plurality of CSB models before applying the probabilistic models.

10. The virtual physiology ecosystem of claim 9, wherein the plurality of CSB models are generalized ordinary differential equation models of physiological systems with shared variables.

11. The virtual physiology ecosystem of claim 5, further comprising a data acquisition device, wherein the data acquisition device to provide the limited data streams from the subject.

12. The virtual physiology ecosystem of claim 11, wherein the local computing device is comprised within the data acquisition device.

13. The virtual physiology ecosystem of claim 11, wherein the data acquisition device can display the real time physiological parameters of the subject to the subject.
